(19) United States
(12) Patent Application Publication
(10) Pub. No.: US 2026/0160581 A1
(43) Pub. Date: Jun. 11, 2026

(54) APPARATUS FOR PERFORMING AN EXAMINATION OF AN OBJECT BY MEANS OF X-RAY RADIATION

(71) Applicant: Siemens Healthineers AG, Forchheim (DE)

(72) Inventors: Bernhard GEBERT, Hagenau (DE); Matthias BERNER, Herzogenaurach (DE); Maurizio BIANCONI, Muenchen (DE); Georg WEBER, Fuerth (DE)

(73) Assignee: Siemens Healthineers AG, Forchheim (DE)

(21) Appl. No.: 19/408,438

(22) Filed: Dec. 4, 2025

(30) Foreign Application Priority Data
Dec. 6, 2024 (DE) 10 2024 211 679.3

(51) Int. Cl. G01D 5/20; A61B 6/04

(52) U.S. Cl. CPC G01D 5/20; A61B 6/0407

(57) ABSTRACT

A functional component is arranged in the radiation region, configured to move in a movement direction and has at least one conductive region. A position sensor has an electrical coil arrangement configured to provide a summation voltage based on sensing of the at least one conductive region. The electrical coil arrangement includes an exciter coil configured to provide an alternating magnetic field, and two series-connected sense coils configured to sense at least part of the alternating magnetic field. The sense coils and the exciter coil are arranged such that the summation voltage across the sense coils depends on a position of the functional component relative to the position sensor. The apparatus is configured to determine a position signal based on the summation voltage, the position signal relating to the position of the functional component relative to the position sensor.

### CROSS-REFERENCE TO RELATED APPLICATION(S)

The present application claims priority under 35 U.S.C. § 119 to German Patent Application No. 10 2024 211 679.3, filed Dec. 6, 2024, the entire contents of which is incorporated herein by reference.

### FIELD

One or more example embodiments of the present invention relate to an apparatus for performing an examination of an object via X-ray radiation, having an X-ray source for emitting X-ray radiation, an X-ray detector for detecting at least some of the X-ray radiation emitted by the X-ray source, a radiation region formed between the X-ray source and the X-ray detector, an object support for holding the object, which object support, for the purpose of performing the examination, can be arranged in the radiation region, an analysis unit, which is coupled to the X-ray detector, for analyzing a radiation signal from the X-ray detector, in particular in order to examine the object, a functional component, which is arranged in the radiation region such that it can move in a movement direction and which has an electrically and/or magnetically conductive region, and a position sensor, in particular for sensing a position of the functional component.

In addition, one or more example embodiments of the present invention relate to a method for performing an examination of an object via X-ray radiation, wherein an X-ray source emits at least some of the X-ray radiation to a radiation region formed between the X-ray source and an X-ray detector, wherein the X-ray detector detects at least some of the X-ray radiation emitted by the X-ray source and emits a radiation signal on the basis of the detected X-ray radiation, wherein the object is held by an object support, which, for the purpose of performing the examination, is arranged in the radiation region, wherein the radiation signal from the X-ray detector is analyzed by an analysis unit coupled to the X-ray detector, wherein a functional component, which has an electrically and/or magnetically conductive region, is moved in the radiation region in a movement direction, wherein a position signal is determined in particular, which relates to a position of the functional component relative to a position sensor.

### BACKGROUND

Apparatuses of the type in question that use X-ray radiation to examine objects, and also methods of the type in question, are well known in the prior art, and therefore, in principle, no separate written evidence is needed. Apparatuses of the type in question, for instance computed tomography devices, X-ray devices, or the like, are used to perform an examination on the object in order to determine a structure of the object at least in part. Such examinations are often employed in materials testing, but also in the field of medical diagnostics, for instance making a diagnosis for a biological material, living organism, or the like. The object can accordingly be a product of an industrial manufacturing process, but also a mining by-product, a body of a living organism, or the like.

Tomography is, amongst other things, an imaging method that is capable of depicting an object in slices, for example. Tomography can be used to determine internal spatial structures and to produce, for example, cross-sectional images. In particular, computed tomography is an imaging method in radiology. X-ray radiation is often employed for this purpose. For example, the X-ray radiation is directed onto the object from different directions and detected by the X-ray detector. By analyzing the radiation signals, it is possible, for example, to ascertain absorption values of the X-ray radiation through the object, which allow the structure of the object to be determined. The analysis unit can be a component of the apparatus or it can also be connected as a separate unit to the apparatus. As a result of the analysis can be provided, for example, imaging relating to the structure of the object.

The functional component can be used to influence the functioning of the apparatus, in particular while the examination is being carried out. Hence, for example, the functional component can be a collimator or the like, for instance in order to be able to influence the X-ray radiation in the radiation region. The functional component can also be used, however, to sense examination parameters while the examination is being carried out, for instance a temperature of the object, scattered radiation, and/or the like. The functional component is usually, at least while the examination is being carried out, partially exposed to the X-ray radiation in the radiation region. To allow adjustment of the action of the functional component as required, the functional component can be moved in a movement direction, which can preferably be a longitudinal direction. A separate drive can be provided for this purpose, which can be suitably controlled by a control unit of the apparatus so that the functional component can be moved to the desired position.

In order to ascertain the position of the functional component, the apparatus has a position sensor, which is used to sense the position of the functional component and to transmit a corresponding position signal to the control unit. In many uses, this results in the position sensor likewise being exposed at least partially to the X-ray radiation. This means that particular demands must be placed on the design of the position sensor in order to be able to achieve the greatest possible accuracy in determining the position while having high radiation resistance, in particular to X-ray radiation. In this context, DE 10 2022 206 622 A1, for example, discloses an apparatus for accommodating a beam-path component for X-ray radiation and a method for providing position information.

In particular, if the functional component has, for example, a filter, collimator, or the like, the positioning of these functional components in the radiation region is meant to be performed with great accuracy in order to be able to achieve as accurate an examination result as possible. In particular, a position of the functional component at any one time must be monitored in order to be able to identify an unwanted change or variation even without radiation. Therefore driving the functional component to perform the movement in the movement direction is normally performed by one or more stepper motors as the drive, the positioning of which can be detected by traveling to one or more end stops and calibrating the encoding as a result. In the case of a stepper motor, the end stops can be identified, for example, by sensing a motor current or by suitable encoding mechanisms, photoelectric sensors, switches, and/or the like. Standard encoding mechanisms work via slotted disks and photoelectric sensors or else via magnetic disks and Hall sensors, for example. Such position sensors, however, prove to have only limited resistance to X-ray radiation and moreover are generally sensitive to mechanical tolerances, which can arise, for example, as a result of rotation in computed tomography (CT) or during assembly or adjustment in manufacture. Furthermore, the use of electronic circuit arrangements, in particular program-controlled computer units, proves to be limited in terms of reliability and a life expectancy that is limited by the X-ray radiation. Therefore, in the prior art, position sensors often have to be shielded against X-ray radiation, as DE 10 2022 206 622 A1 also discloses.

It also proves to be a disadvantage that an absolute position or a reference position of the position sensor cannot be sensed directly in the prior art, and potential discrepancies can lead to complex errors in examination results, which sometimes can only be rectified with difficulty. Moreover, the reliability, in particular in relation to aging, of the position sensor is limited.

### SUMMARY

An object of one or more example embodiments of the present invention is to develop an apparatus and a method such that radiation resistance of the position sensor can be improved and/or radiation-induced aging of the position sensor can be reduced.

An apparatus and a method as claimed in the independent claims are proposed as a solution to address this object. Features of the dependent claims provide advantageous developments.

Independent of the grammatical term usage, individuals with male, female or other gender identities are included within the term.

With regard to an apparatus of the type in question is proposed in particular that the position sensor has an electrical coil arrangement for sensing the conductive region and for providing a position signal on the basis of the sensing of the conductive region, wherein the coil arrangement has an exciter coil for providing an alternating magnetic field, and two series-connected sense coils for sensing at least part of the alternating magnetic field, wherein the sense coils and the exciter coil are arranged such that a summation voltage across the two sense coils depends on a position of the functional component relative to the position sensor, wherein the apparatus is designed to determine on the basis of the summation voltage a position signal, which relates to the position of the functional component relative to the position sensor.

With regard to a method of the type in question, it is proposed in particular that an electrical coil arrangement of a position sensor is used to sense the conductive region, and a summation voltage is provided on the basis of the sensing of the conductive region, wherein an alternating magnetic field is provided by an exciter coil of the coil arrangement, wherein at least part of the alternating magnetic field is sensed by two series-connected sense coils of the coil arrangement, wherein the sense coils and the exciter coil are arranged such that the summation voltage across the two sense coils depends on a position of the functional component relative to the position sensor, wherein a position signal, which relates to the position of the functional component relative to the position sensor, is determined on the basis of the summation voltage.

The solution is based, amongst other things, on the idea that position sensing can be realized reliably via the position sensor without the position sensor needing to have moving parts. This has the advantage that, for example, mechanical wear in the position sensor essentially need not occur in intended use over the predicted operating life. The use of the position sensor using the coil arrangement also proves advantageous because the coil arrangement can easily be made particularly radiation-resistant.

At the same time, the use of the magnetic field as the medium for sensing the position allows a physical decoupling from the X-ray radiation, so that the position sensor can achieve a position-sensing result that essentially cannot be affected by the X-ray radiation. By virtue of using the coil arrangement, it is possible to design the position sensor such that only the coil arrangement needs to be located in the radiation region, and therefore all the further components of the position sensor that are required for operating the coil arrangement and for determining the position can be located essentially outside the radiation region. Separate costly shielding of these components of the position sensor can thus be reduced or entirely eliminated.

At the same time, the coil arrangement makes it possible to sense the position of the functional component reliably, specifically because the summation voltage depends on the position of the functional component, in particular of the electrically and/or magnetically conductive region. In addition, there is also no need here for any mechanical contact between the functional component and the position sensor. Furthermore, there is also no need to travel to an end stop, because the current position of the functional component can be ascertained reliably just from the summation voltage. The conductive region affects the alternating magnetic field, which is sensed by the sense coils, in terms of its geometry. It is thereby possible to ascertain a unique relationship between the summation voltage, which depends on the induced voltages in the sense coils, and the position of the functional component.

The sense coils are preferably arranged side by side along the movement direction. The sense coils can preferably be substantially identical in terms of their geometric dimensions. The sense coils are preferably flat coils and each has a winding that has at least one, preferably a plurality of turns. Particularly preferably, the windings of the sense coils have the same number of turns. For example, the sense coils can be substantially in the form of an Archimedean spiral. A cross-section of the sense coils and/or of the exciter coil can be substantially round or else angular. The coils can be in the form of cylindrical coils, flat coils, Archimedean spirals and/or the like. In particular, the exciter coil can have a different geometry from the sense coils.

The position sensor can have a single coil arrangement. It is also possible, however, that the position sensor has a plurality of coil arrangements depending on the design of the apparatus. It can be provided that the coil arrangements are arranged along the movement direction of the functional component substantially adjacent to each other. This means that even particularly large movement travels can be sensed by the position sensor.

The conductive region can be formed, for example, from an electrically conductive material such as metal, a metal alloy and/or the like. Alternatively or additionally, however, the conductive region can also have a magnetizable material, for example a ferrite, a magnetizable metal, a magnetizable alloy and/or the like. The conductive region is preferably connected as a separate member to the functional component. It can also be integral with the functional component, however. The conductive region is preferably arranged in the apparatus in such a way that it can be exposed to the alternating magnetic field emitted by the exciter coil. The sense coils can accordingly sense the alternating magnetic field from the exciter coil. For this purpose, a substantially constant alternating voltage or a substantially constant alternating current is applied to the exciter coil so that the exciter coil accordingly provides the alternating magnetic field.

The X-ray detector serves in particular to detect secondary X-ray radiation that is at least partially transmitted through the object, partially absorbed, and/or at least partially scattered by the object while the examination is being carried out, and to provide on the basis of the detected secondary X-ray radiation a detector signal as the radiation signal. The detector signal can be analyzed by the analysis unit in order to be able to determine the structure of the object at least in part. For example, M. H. Yaffe and H. A. Rowlands disclose in X-Ray Detectors for Digital Radiography, in Phys. Med. Biol. 42(1997 ), pages 1 to 39, X-ray detectors such as can be used in X-ray devices and methods of the type in question.

Apparatuses, in particular X-ray devices, of the type in question can have an extremely wide variety of designs. An X-ray device that is suitable for X-ray tomography proves to be particularly advantageous, in which digital cross-sectional images showing the structure of the object are determined from absorption values of X-ray radiation through the object, for example acting on the object from different directions. In conventional X-ray methods, it is usual to expose the object under examination to the X-ray radiation, to receive the secondary X-ray radiation via an X-ray detector, and to analyze the radiation signal via the X-ray analysis unit. The radiation signal is preferably an electrical signal, for example an analog or digital electrical signal. The analysis unit can have for the purpose of analysis an electronic hardware circuit and/or a program-controlled computer unit. The X-ray analysis unit can also have interfaces for providing the analysis and/or output devices for outputting the analysis.

It is also possible in computed tomography to produce, for example, absorption profiles of the object from a plurality of spatial directions. The structure of the object can likewise be determined therefrom. For example, computer-aided image reduction can be used to determine a specific absorption coefficient for a certain volume element of the object, and the spatial structure of the object can be determined in this way. X-ray tomography is an imaging method that can be used, for example, to depict a slice inside the object under examination.

It is also proposed to select a winding of each sense coil such that induced voltages in the series-connected sense coils cancel out when the functional component is in a defined reference position relative to the position sensor. This can be achieved, for example, by a winding sense of the windings of the sense coils being inverted with respect to each other. As a result, the induced voltages can cancel out when the sense coils are exposed to the same magnetic flux of the alternating magnetic field. This can be provided in a specific position of the functional component. In principle, it can also be provided that full cancellation of the induced voltages is not realized. There is also the option that the cancellation is identified by a local minimum of the summation voltage. Therefore in the reference position, the summation voltage is substantially around zero. It can, however, also have just a local minimum in the region of the reference position.

In addition, it is proposed that the sense coils are arranged adjacent to each other in a plane that is parallel to the movement direction. The position sensor function can be realized particularly easily in this way, because two sense coils arranged side by side can be exposed to the same alternating magnetic field. The exciter coil is preferably also arranged in the same plane.

In principle, the exciter coil can likewise be a flat coil, in which case the exciter coil preferably surrounds the sense coils.

It proves particularly advantageous when the series-connected sense coils are coupled to a sensing-side electrical capacitor in order to form a sensing-side resonant circuit. It can be provided that the sensing-side capacitor is connected to the series circuit. It can also be provided, however, that one capacitor is connected to each of the sense coils. In the case of a plurality of coil arrangements, the resonant circuits formed preferably have the same resonant frequency. This can significantly increase the sensitivity of the position sensor. The capacitor is preferably a ceramic capacitor, which has proved particularly resistant to X-ray radiation. In principle, however, the capacitor can also be formed by suitable conductive surfaces, which can be opposite each other on a circuit carrier, in order to be able to provide the relevant electrical capacitance.

It is further proposed that the sense coils are arranged on a circuit carrier, which provides a connection region that is located outside the radiation region, wherein preferably at least part of the coil arrangement is located in the radiation region. This is a particularly simple way to realize the sense coils. The circuit carrier can be, for example, a printed circuit board or else another insulation material in the form of a panel, on which the sense coils can be arranged. For example, the sense coils can be in the form of conductor tracks on the circuit carrier. The circuit carrier can be formed, for example, from a suitable material, for instance a plastic or ceramic, in particular FR4 for example. Particularly preferably, the exciter coil is also arranged on the circuit carrier. The entire coil arrangement can thereby be provided as a module that can be handled as a single unit.

The circuit carrier is preferably designed such that the coil arrangement can be located in the radiation region. At the same time, the circuit carrier can provide the connection region, which is preferably located outside the radiation region. Hence further components of the position sensor that are meant to be connected to the coil arrangement can be positioned outside the radiation region, with the result that separate shielding against the X-ray radiation can be reduced or avoided.

Preferably, the exciter coil can also be connected to an exciter-side electrical capacitor, so that an exciter-side resonant circuit can be formed. The resonant frequency of the exciter-side resonant circuit preferably corresponds to the resonant frequency of the resonant frequency formed by the sense coils and their capacitor.

It can be provided in particular that the sense coils are arranged on the surface of a planar circuit carrier. The planar circuit carrier can be a printed circuit board, for example.

It proves particularly advantageous if the sense coils are arranged on a first surface of the circuit carrier, and the exciter coil is arranged on a second surface of the circuit carrier, which second surface differs from the first surface and is opposite the first surface. It is thereby possible to achieve galvanic isolation between the sense coils and the exciter coil. At the same time, reliable functionality in terms of exposure of the sense coils to the alternating magnetic field can be realized by the circuit carrier, in particular if it is very thin. An internal diameter of the exciter coil is preferably selected such that the sense coils can be positioned inside the internal diameter.

It is additionally proposed that the series-connected sense coils are coupled to a sensing-side electrical capacitor in order to form a sensing-side resonant circuit, wherein an alternating current and/or an alternating voltage is applied to the exciter coil by a frequency generator coupled to the exciter coil, wherein a frequency of the alternating current or of the alternating voltage approximately corresponds to a resonant frequency of the sensing-side resonant circuit. This can achieve a particularly high sensitivity of the position sensor with regard to sensing the position of the functional component.

It has been found particularly advantageous to select as the resonant frequency a frequency in a range of approximately 800 kHz to approximately 1.6 MHz. It has been found that especially low interference occurs in this frequency range, allowing a further improvement in the function of the position sensor.

It is further proposed that the position signal and a processing signal, which has a processing frequency which in particular is greater than the resonant frequency, are mixed by a first mixer unit in order to provide an intermediate frequency signal. The providing of the intermediate frequency signal allows improved signal processing, in particular in terms of the accuracy of the sensing of the position of the functional component. The processing frequency is a definable frequency, which can be fixed for a specific use, for example. It can also be provided that the definition of the processing frequency is changed or varied depending on a specific event. Although the processing frequency can be chosen in principle to be less than the resonant frequency, the processing frequency can preferably be greater than the resonant frequency. Particularly preferably, the processing frequency can be defined depending on the resonant frequency. It is thereby possible to provide the intermediate frequency signal with a substantially fixed frequency, so that subsequent signal analysis units can be adapted specifically to the intermediate frequency signal. This can improve the signal analysis. Both up-mixing and down-mixing can be achieved.

In addition, it is proposed that the intermediate frequency signal is filtered by an intermediate frequency band-pass filter in order to provide an intermediate frequency filtered signal. Interference and unwanted influences can be filtered out particularly advantageously thereby, allowing a further improvement in the reliability and function, in particular in terms of the accuracy, of the position sensor. It proves particularly advantageous if the intermediate frequency signal can be provided at a substantially constant frequency. As a result, the intermediate frequency band-pass filter can be adapted specifically to this frequency, whereby the desired filter action can be improved.

It is further proposed to perform time-discrete sampling of the intermediate frequency signal, the intermediate frequency filtered signal and/or the summation voltage. As a result, it is possible to input the intermediate frequency signal, the intermediate frequency filtered signal and/or the sensor signal to digital signal processing. The digital signal processing can be performed by a program-controlled computer unit, for example a digital signal processor (DSP), an FPGA, an ASIC, and/or the like. Once the sensor signal has undergone time-discrete sampling, the subsequent further signal processing can be performed almost entirely in digitized form.

According to a development, it is proposed that the intermediate frequency signal or the intermediate frequency filtered signal on one side and a difference frequency signal on the other side are mixed by a second mixer unit in order to provide a baseband signal, wherein a frequency of the difference frequency signal substantially corresponds to a difference in the frequencies of the position signal and the processing signal. This procedure can achieve a form of demodulation, which makes it possible to provide the baseband signal which is able to serve as the position signal practically unchanged. Of course, the position signal can be provided on the basis of the baseband signal, wherein further additional processing steps can be provided according to need and depending on the design of the apparatus or implementation of the method.

It is further proposed that the difference frequency signal has a frequency of approximately 8.5 kHz to approximately 50 kHz. This frequency range has proved particularly advantageous for the signal processing as part of the position sensing.

In addition, it is proposed that the baseband signal is filtered by a low-pass filter in order to provide the position signal. As a result, a further improvement in the position signal can be achieved in terms of accuracy and reliability.

Advantages and effects stated for the apparatus according to one or more example embodiments of the present invention apply equally also to the method according to one or more example embodiments of the present invention, and vice versa. In particular, method features can therefore also be worded as apparatus features, and vice versa. The features and combinations of features stated in the description above and also the features and combinations of features stated in the following description of exemplary embodiments and/or shown in the figures alone can be used not only in the respectively stated combination, but also in other combinations. The present invention shall therefore be deemed to include or disclose also embodiments that are not described and shown explicitly in the figures but can be derived and produced from the described embodiments by separate feature combinations.

The features, functions and/or effects presented with reference to the exemplary embodiments can each separately constitute individual features, functions and/or effects to be considered independently of one another and which each develop the present invention also independently of one another. Therefore the exemplary embodiments are intended to include also other combinations than those described in the embodiments. In addition, further features, functions and/or effects of the present invention that have already been described can also be added to the described embodiments.

### BRIEF DESCRIPTION OF THE DRAWINGS

In the figures:

FIG. 1 shows a schematic representation of an x-ray device;

FIG. 2 shows in a schematic cross-sectional diagram a segment of a first embodiment of a position sensor of the apparatus shown in FIG. 1;

FIG. 3 shows in a schematic plan view a segment of a second embodiment of the position sensor of the apparatus shown in FIG. 1;

FIG. 4 shows a schematic circuit diagram of the position sensor shown in FIG. 3;

FIG. 5 shows a schematic diagram of a graph of a summation voltage of two series-connected sense coils of the coil arrangement shown in FIG. 4 as a function of a position of a collimator of the apparatus shown in FIG. 1; and

FIG. 6 shows a schematic block diagram of the position sensor shown in FIG. 3.

### DETAILED DESCRIPTION

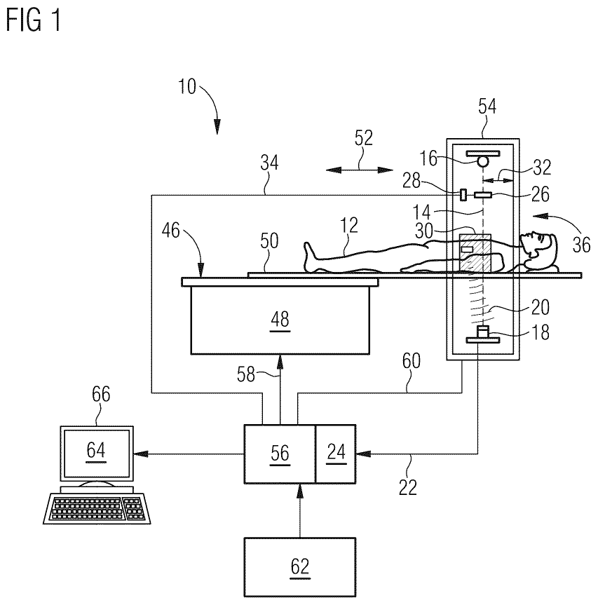

FIG. 1 shows a schematic representation of an X-ray device 10 as an apparatus for performing an examination of an object via X-ray radiation, which in the present case is an X-ray computed tomography (CT) device. The X-ray device 10 is used, for example, to perform an X-ray examination on a patient. The X-ray device 10 is not confined to the use for patients, however, and can in principle be used for any objects, for example in materials testing or the like.

The X-ray device 10 has a patient positioning apparatus 46, which in turn has a displacement table 48 and a patient couch 50, which can travel relative to the displacement table 48 in a longitudinal direction 52, as the object support for holding the object, in this case a patient. The patient positioning apparatus 46 can be used to position the patient 12, located on the patient couch 50, in a longitudinal direction 52 in a through-hole of a gantry 54. The gantry 54 is an annular support structure in which the through-hole is formed in the shape of a tunnel, so that an examination region 30 of the patient 12 can be introduced into the through-hole for the purpose of performing the X-ray examination. The displacement table 48 can be used to position the patient in the through-hole of the gantry 54 in a defined manner, so that not only can the examination region 30 be well exposed to the X-ray radiation 14 but also secondary X-ray radiation 20 can be detected effectively by the X-ray detector 18.

In an annular region of the gantry 54 surrounding the through-hole is arranged an X-ray source 16, which is used to emit the X-ray radiation 14.

Radially opposite is arranged in the annular region the X-ray detector 18. DE 10 2008 050 838, for example, describes the function, arrangement and design of a suitable X-ray detector, and therefore reference is made to the statements therein for additional information on this subject.

As FIG. 1 shows, the X-ray device 10 additionally has a control unit 56, which provides a control signal 58, by which the patient couch 50 can be positioned in the gantry 54. In addition, the control unit 56 provides a control signal 60, which, inter alia, is used to control the X-ray source 16 and the X-ray detector 18 in the gantry 54.

The X-ray device 10 furthermore has a user interface 62, which can be used to define the control signals 58, 60 at least in part. The user interface 62 can have, for example, a joystick, a computer mouse, a keyboard, combinations thereof and/or the like.

The examination region 30 of the patient 12 is examined by moving the patient 12 via the patient couch 50 into the gantry 54 in a longitudinal direction 52. During this movement, it is preferably provided that the X-ray source 16 and the X-ray detector 18 move synchronously around the patient 12 in a circular movement. The X-ray source 16 emits during this process the X-ray radiation 14, which penetrates the examination region 30 of the patient 12. The X-ray radiation 14 is partially absorbed, scattered and/or deflected in the examination region 30 of the patient 12, with the result that the secondary X-ray radiation 20 exits on the opposite side of the examination region 30. The secondary X-ray radiation 20 can be detected by the X-ray detector 18. The X-ray detector 18 provides corresponding detector signals 22 as radiation signals, which are input to the X-ray analysis unit 24 as the analysis unit. This performs corresponding analysis of the detector signals 22 and transmits analyzed data to the control unit 56. In the control unit 56, this data can be processed further, for instance in order to convert projection images into image information 64. The image information 64 can be transmitted from the control unit 56 to a display unit 66, which facilitates a graphical visual display for a user of the X-ray device 10.

Between the X-ray detector 18 and the X-ray source 16 is formed a radiation region 36. In the radiation region 36 is arranged the examination region 30, for example. In the radiation region 36 is further arranged a collimator 26 as the functional component, which can move in a movement direction 32. For this purpose, the collimator 26 is coupled to a linear drive (not presented further), which can be controlled by the control unit 56. The collimator 26 can be moved into a desired position in the radiation region 36 via the linear drive. The collimator 26 can be used to influence the X-ray radiation in the radiation region 36.

Since information about the position of the collimator 26 is important for performing the examination of the patient 12, the X-ray device 10 has a position sensor 28, which senses the position of the collimator 26 in the radiation region 36 and transmits a corresponding position signal 34 to the control unit 56.

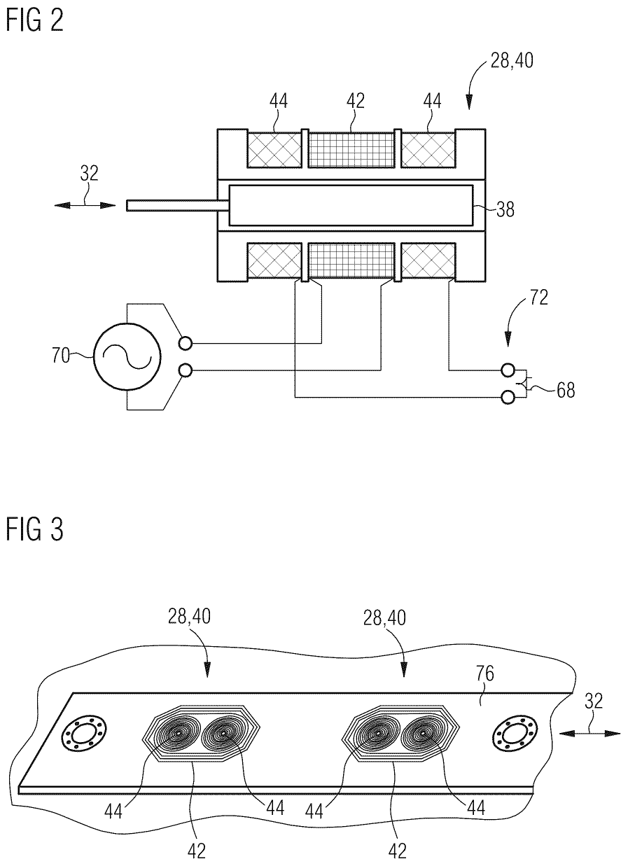

FIG. 2 shows in a schematic circuit diagram a segment of a first embodiment for the position sensor 28. As can be seen from FIG. 2, the position sensor 28 has an electrical coil arrangement 40, which is used to sense a conductive region 38 of the collimator 26. The coil arrangement 40 is additionally used to provide the summation voltage 68 on the basis of the sensing of the conductive region 38.

In the present embodiment, the collimator 26 is mechanically connected via a coupling rod (not shown) to a ferrite body, which forms the conductive region 38. This forms in particular a magnetically conductive region. In alternative embodiments, an electrically conductive region or an electrically and magnetically conductive region can be provided, for example.

FIG. 2 also shows that the coil arrangement 40 has an exciter coil 42, which in the present case is a cylindrical coil and is connected to a generator 70 so that in intended use, an alternating current, which substantially has a constant amplitude and a constant frequency, can be applied to the exciter coil 42. In the present case, the generator 70 can be controlled by the control unit 56, so that at least the amplitude or the frequency of the alternating current can be adjusted. It is also possible, however, to apply a corresponding alternating voltage to the exciter coil 42.

FIG. 2 also shows that sense coils 44 are arranged in an axial direction, each adjacent to one axial end of the exciter coil 42. The sense coils 44 are likewise cylindrical coils and have substantially the same internal diameter as the exciter coil 42. The sense coils 44 are positioned coaxial with the exciter coil 42. The sense coils 44 are connected electrically in series and in the present case have the same number of turns but a winding sense that is inverted in relation to each other. This results in a summation voltage 68 across the two sense coils 44 that, in particular with regard to the amplitude, corresponds to a difference between the voltages induced in each of the individual sense coils. As a result of the applied alternating current, the exciter coil 42 provides an alternating magnetic field, which flows, inter alia, through the sense coils 44.

In accordance with the design of the sense coils 44 and the electrical interconnection, in the position shown in FIG. 2 for the conductive region 38 is obtained a summation voltage 68 that amounts to approximately zero. The voltages induced in each into the individual sense coils 44 thus cancel out. As soon as the collimator 26, and accordingly the conductive region 38, moves in the movement direction 32, the coupling of the sense coils 44 with respect to the exciter coil 42 changes, and therefore the amplitudes of the voltages induced in each of the individual sense coils 44 differ from each other and consequently a summation voltage 68 is provided that deviates significantly from zero. An amplitude of the summation voltage 68 and/or a phase with respect to the alternating current of the generator 70 can be associated with a position of the conductive region 38 and hence also with the position of the collimator 26. It is thereby possible for the position signal 34, which is determined on the basis of the summation voltage 68, to contain accurate position information relating to the conductive region 38 and hence relating to the collimator 26.

In the present case, the coil arrangement 40 is located close to the collimator 26, i.e. in the radiation region 36. Provision is also made for a connection region 72, at which the summation voltage 68 is provided. In the present case, the connection region 72 is located outside the radiation region 36. As a result, a corresponding circuit arrangement 80 for processing the summation voltage 68 can likewise be located outside the radiation region 36. The present design of the position sensor 28 has the advantage that no mechanically moving parts need to be provided there. Furthermore, the design of the position sensor 28 proves to be particularly radiation resistant. At the same time, the position of the collimator 26 can be sensed with high accuracy.

FIG. 3 shows a second embodiment for a position sensor 28, which in principle can be arranged instead of the position sensor 28 in the X-ray device 10 shown in FIG. 1. With regard to this embodiment, reference is additionally made to the statements relating to the previous embodiment. Only the differences from the first embodiment of the position sensor 28 are described below.

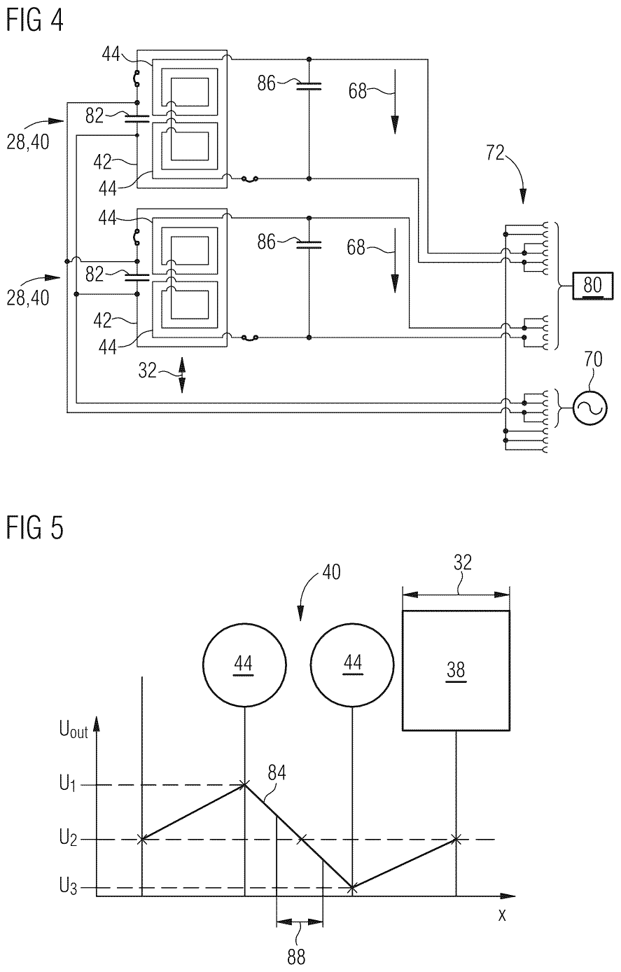

As FIG. 3 shows, two coil arrangements 40 are arranged on the printed circuit board 76 adjacent to each other in the movement direction 32 of the conductive region 38. The coil arrangements 40 are substantially identical in the present case. FIG. 4 shows in relation to this a schematic circuit diagram of the position sensor 28.

In the present embodiment, the coil arrangement 40 is formed by conductor tracks on the printed circuit board 76. This allows the coil arrangement 40 to have a particularly flat, i.e. space-saving, and low-cost implementation. The coils 42, 44 are arranged substantially approximately in the same plane, which is defined by the printed circuit board 76. The coil arrangement 40 has an exciter coil 42, to which is connected in parallel an exciter-side capacitor 82. As a result, the exciter coil 42 forms with the exciter-side capacitor 82 an exciter-side resonant circuit, which in the present case has approximately the same resonant frequency, which also substantially corresponds to the frequency of the alternating current provided by the generator 70. In the present embodiment, it can optionally be provided that the exciter coil 42 and the sense coils 44 of each coil arrangement 40 are arranged on different, opposite surfaces of the printed circuit board 76.

As FIG. 4 also shows, the two sense coils 44 of the coil arrangement 40 are connected in series, with the sense coils 44 having the same number of turns but an inverted winding sense. The sense coils 44 are formed inside an internal diameter of the exciter coil 42. The sense coils are arranged adjacent to each other in the movement direction 32. In the present embodiment, all the sense coils 44 and the exciter coils 42 are arranged adjacent to each other parallel to the movement direction 32.

The summation voltage 68 is dropped across the two sense coils 44 of the coil arrangement 40. The summation voltage 68 can be input to the circuit arrangement 80 of the position sensor 28 via a connection region 72, which can optionally be formed on the printed circuit board 76. The circuit arrangement 80 analyzes the summation voltage 68 and provides the position signal 34 on the basis thereof.

In addition, the generator 70 is also connected to the exciter coil 42, so that the alternating current can be applied to the exciter coil 42. In this embodiment, two exciter coils 42 are connected in parallel to the generator 70.

In this embodiment, the conductive region 38 is situated in the movement direction 32 in the region of one of the coil arrangements 40, and therefore the position of the conductive region 38, and hence also the position of the collimator 26, can be ascertained accurately from the summation voltage 68 of the corresponding coil arrangement 40. The adjacent arrangement of the coil arrangements 40 is defined by the movement direction 32. The two position sensors 28 on the same printed circuit board 76 can be used, for example, to detect two limits of travel of the collimator 26 along the movement direction 32.

It is also evident from the second embodiment of the position sensor 28 that a number of coil arrangements can be selected depending on the travel of the conductive region 38. Of course, dimensions of the coil arrangements 40 themselves can also be adapted as required. In addition, the geometry of the respective coils 42, 44 can be varied as required; for instance, the coils 42, 44 can be circular, angular, in particular rectangular, or the like. In particular, the sense coils 44 can also be in the form of Archimedean spirals.

According to another embodiment, two diaphragm jaws located opposite each other can be provided, between which is formed a diaphragm slit for the X-ray radiation 14, and which can be moved relative to each other along the movement direction 32 in order to adjust a width of the diaphragm slit. In this case, one of the two position sensors 28, which are arranged on the same printed circuit board 76, senses the position of one of the two diaphragm jaws, and the other of the two position sensors 28, which are arranged on the same printed circuit board 76, senses the position of the other of the two diaphragm jaws.

FIG. 4 also shows that a series circuit of the two sense coils 44 of a coil arrangement 40 is connected in parallel with a sensing-side capacitor 86. This results in a sensing-side resonant circuit, the resonant frequency of which is tuned to the resonant frequency of the exciter coil 42 with the capacitor 82. This arrangement can achieve high sensitivity.

In the present embodiment, the frequency of the alternating current from the generator 70 corresponds to approximately 1 MHz. Despite this comparatively high frequency, it is possible to achieve high sensitivity and resolution of the position using the position sensor shown.

FIG. 5 shows in a schematic diagram a possible variation in the position signal 34, which in the present case is represented by an electrical voltage, when the conductive region 38 is moved in the movement direction 32. The voltage variation is characterized by a graph 84. If the conductive region 38 is outside the sense coils 44, a voltage U2 is established. As soon as the conductive region 38 comes within range of a first sense coil of the particular coil arrangement 40, the voltage falls to a value U3. At this point, the conductive region 38 has reached approximately the center point of the first sense coil 44. If the conductive region 38 is moved further, the voltage rises until the conductive region 38 reaches the center point of the second sense coil 44 of the coil arrangement 40. The voltage corresponds to U1 in this position. If the conductive region 38 is again moved further, the voltage falls from the value U1 to the value U2, at which the conductive region 38 has again left the coil arrangement 40.

The coil arrangement 40 proves particularly sensitive in a region 88, which lies between the two sense coils 44. In this region, the position of the conductive region 38 can be detected with particularly high accuracy.

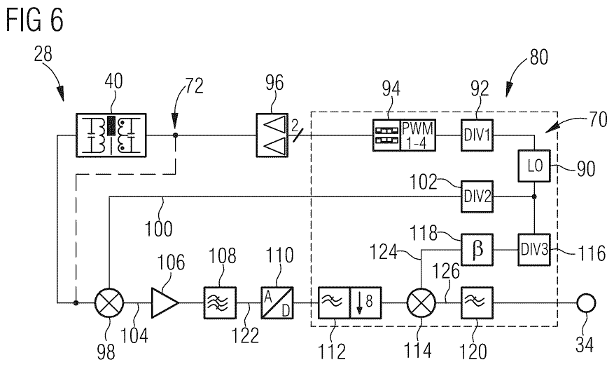

FIG. 6 shows in a schematic block diagram a design of the circuit arrangement 80, which is connected to the connection region 72.

FIG. 6 shows that the generator 70 comprises an oscillator 90. The oscillator 90 provides an alternating voltage, which is reduced to a desired sub-frequency by a frequency divider 92. A selection unit 94 is used to set the desired frequency, which in the present case corresponds to approximately 1 MHz. If required, however, the frequency can also be varied in the present case in steps of approximately 50 kHz from 800 kHz to 1.5 MHz. A suitable alternating current is provided for the exciter coil 42 at the connection region 72 via an amplifier 96.

The summation voltage 68 is input via the connection region 72 to a first mixer 98 of the circuit arrangement 80. The first mixer 98 mixes the summation voltage 68 with a processing signal 100. The processing signal 100 is provided from the alternating voltage of the oscillator 90 by a frequency divider 102 of the circuit arrangement 80. The processing signal 100 has a frequency that in the present case is greater than the frequency of the alternating current from the generator 70 by approximately 10 kHz. Consequently, the mixing by the first mixer 98 results in an intermediate frequency signal 104 which has a frequency of approximately 10 kHz.

An amplifier 106 of the circuit arrangement 80 amplifies the intermediate frequency signal 104. Then, the intermediate frequency signal 104 is filtered by a band-pass filter 108 of the circuit arrangement 80. After the band-pass filter 108, or more specifically the intermediate frequency band-pass filter, exists an intermediate frequency filtered signal 122, which is input to an analog-to-digital converter 110 of the circuit arrangement 80. In the present case, the intermediate frequency signal is oversampled as part of the analog-to-digital conversion. The further signal processing is performed digitally.

The signal digitized in this way is downsampled in 112, and the signal produced in this way is input to a second mixer 114 of the circuit arrangement 80.

Also input to the second mixer 114 is a difference frequency signal 124, which is produced from the alternating voltage provided by the oscillator 90, namely by providing this alternating voltage via a further frequency divider 116 of the circuit arrangement 80 and via a delay unit 118 of the circuit arrangement 80. A frequency of the difference frequency signal 124 approximately corresponds to a difference in the frequencies of the summation voltage 68 and the processing signal 100. In the present case, the difference frequency signal 124 has a frequency of approximately 10 kHz. The frequency can also vary according to need, however, for example from approximately 8.5 kHz to 11.5 kHz.

The second mixer 114 provides a baseband signal 126 as the result of the mixing. Finally, the baseband signal 126 is filtered by a low-pass filter 120 of the circuit arrangement 80 to provide the position signal 34.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. An apparatus for performing an examination of an object via X-ray radiation, the apparatus comprising:
an X-ray source configured to emit X-ray radiation;
an X-ray detector configured to detect at least some of the X-ray radiation emitted by the X-ray source;
a radiation region formed between the X-ray source and the X-ray detector;
an object support configured to hold the object, the object support configured to be arranged in the radiation region for performing the examination;
an analysis unit coupled to the X-ray detector, the analysis unit configured to analyze a radiation signal from the X-ray detector;
a functional component arranged in the radiation region, the functional component configured to move in a movement direction and having at least one conductive region, wherein the at least one conductive region includes at least one of an electrically conductive region or a magnetically conductive region; and
a position sensor having an electrical coil arrangement configured to
sense the at least one conductive region, and
provide a summation voltage based on sensing of the at least one conductive region, wherein the electrical coil arrangement includes an exciter coil configured to provide an alternating magnetic field, and two series-connected sense coils configured to sense at least part of the alternating magnetic field,
the two series-connected sense coils and the exciter coil are arranged such that the summation voltage across the two series-connected sense coils depends on a position of the functional component relative to the position sensor, and
the apparatus is configured to determine a position signal based on the summation voltage, the position signal relating to the position of the functional component relative to the position sensor.

2. The apparatus as claimed in claim 1, wherein a respective winding of each of the two series-connected sense coils is selected such that voltages induced in each individual sense coil cancels out when the functional component is in a defined reference position of the functional component relative to the position sensor.

3. The apparatus as claimed in claim 1, wherein the two series-connected sense coils are arranged adjacent to each other in a plane that is parallel to the movement direction.

4. The apparatus as claimed in claim 1, wherein
the position sensor has a sensing-side electrical capacitor, and
the two series-connected sense coils are coupled to the sensing-side electrical capacitor to form a sensing-side resonant circuit.

5. The apparatus as claimed in claim 1, further comprising
a connection region connected in an electrically conductive manner to the two series-connected sense coils, the connection region configured to provide the summation voltage outside the radiation region.

6. The apparatus as claimed in claim 5, wherein the two series-connected sense coils are arranged on a surface of a planar circuit carrier.

7. A method for performing an examination of an object via X-ray radiation, the method comprising:
emitting, by an X-ray source, at least some of the X-ray radiation to a radiation region formed between the X-ray source and an X-ray detector, wherein the object is held by an object support arranged in the radiation region for performing the examination;
detecting, by the X-ray detector, the at least some of the X-ray radiation emitted by the X-ray source;
emitting, by the X-ray detector, a radiation signal based on the at least some of the X-ray radiation;
analyzing, by an analysis unit coupled to the X-ray detector, the radiation signal from the X-ray detector;
moving a functional component in the radiation region in a movement direction, the functional component having at least one conductive region, wherein the at least one conductive region includes at least one of an electrically conductive region or a magnetically conductive region;
sensing, via an electrical coil arrangement of a position sensor, the at least one conductive region;
providing a summation voltage based on sensing of the at least one conductive region;
providing an alternating magnetic field by an exciter coil of the electrical coil arrangement, wherein
at least part of the alternating magnetic field is sensed by two series-connected sense coils of the electrical coil arrangement, and
the two series-connected sense coils and the exciter coil are arranged such that the summation voltage across the two series-connected sense coils depends on a position of the functional component relative to the position sensor; and
determining a position signal based on the summation voltage, the position signal relating to the position of the functional component relative to the position sensor.

8. The method as claimed in claim 7, wherein
the two series-connected sense coils are coupled to a sensing-side electrical capacitor to form a sensing-side resonant circuit, and
at least one of an alternating current or an alternating voltage, having a frequency that matches a resonant frequency of the sensing-side resonant circuit, is applied to the exciter coil by a frequency generator coupled to the exciter coil.

9. The method as claimed in claim 8, wherein the resonant frequency of the sensing-side resonant circuit is at least one of greater than 800 kHz or less than 1.6 MHz.

10. The method as claimed in claim 8, further comprising:
mixing, by a first mixing unit, the summation voltage and a processing signal to provide an intermediate frequency signal; wherein
the processing signal has a processing frequency that is greater than the resonant frequency of the sensing-side resonant circuit.

11. The method as claimed in claim 10, wherein the intermediate frequency signal is filtered by an intermediate frequency band-pass filter to provide an intermediate frequency filtered signal.

12. The method as claimed in claim 11, further comprising:
performing time-discrete sampling of at least one of the intermediate frequency signal, the intermediate frequency filtered signal or the summation voltage.

13. The method as claimed in claim 11, further comprising:
mixing, by a second mixer unit, the intermediate frequency signal or the intermediate frequency filtered signal on one side and a difference frequency signal on another side to provide a baseband signal, wherein
a frequency of the difference frequency signal corresponds to a difference in frequencies of the summation voltage and the processing signal.

14. The method as claimed in claim 13, wherein the frequency of the difference frequency signal is at least one of greater than 8.5 kHz or less than 50 kHz.

15. The method as claimed in claim 13, wherein the baseband signal is filtered by a low-pass filter to provide the position signal.

16. The apparatus as claimed in claim 5, wherein the electrical coil arrangement is located at least partially in the radiation region.

17. The apparatus as claimed in claim 2, wherein the two series-connected sense coils are arranged adjacent to each other in a plane that is parallel to the movement direction.

18. The apparatus as claimed in claim 17, wherein
the position sensor has a sensing-side electrical capacitor, and
the two series-connected sense coils are coupled to the sensing-side electrical capacitor to form a sensing-side resonant circuit.

19. The apparatus as claimed in claim 18, further comprising:
a connection region connected in an electrically conductive manner to the two series-connected sense coils, the connection region configured to provide the summation voltage outside the radiation region.

20. The apparatus as claimed in claim 3, wherein
the position sensor has a sensing-side electrical capacitor, and
the two series-connected sense coils are coupled to the sensing-side electrical capacitor to form a sensing-side resonant circuit.
